(19) United States
(12) Patent Application Publication
(10) Pub. No.: US 2013/0211287 A1
(43) Pub. Date: Aug. 15, 2013

(54) UNIT FOR COLLECTING ANIMAL MATERIAL

(71) Applicants: Johan Decaluwe, Laval (FR); Johan De Meulemeester, De Printe (BE)

(72) Inventors: Johan Decaluwe, Laval (FR); Johan De Meulemeester, De Printe (BE)

(73) Assignee: Allflex Europe, Vitre (FR)

(21) Appl. No.: 13/702,967

(22) PCT Filed: May 19, 2011
(86) PCT No.: PCT/EP2011/058106

§ 371 Date: Apr. 25, 2013

(30) Foreign Application Priority Data
Jun. 9, 2010 (FR) 1054564

(51) Int. Cl. A61B 10/02

(52) U.S. Cl. CPC A61B 10/02
USPC 600/562

(57) ABSTRACT

The invention relates to a unit for taking at least one sample of animal material, made up of a package comprising at least one sampling assembly. According to the invention, such an assembly comprises: a sampling tool; a component for taking said sample; a component for storing said sample; at least one of said sampling or storing components being pre-mounted on at least one support provided for this purpose on said sampling tool.

### 1. FIELD OF THE INVENTION

The field of the invention is that of controls on animals, or animal-derived products, performed by collecting animal material.

The term “animal material” is understood herein to mean any material that can be collected from a living or dead animal, possibly after transformation, such as a tissue sample, a blood sample, a bone-marrow sample, a sample of viscera, a portion of the animal intended for consumption, a piece of leather, etc.

More specifically, the invention pertains to the means for performing such a collection and for packaging them.

### 2. PRIOR ART

There are many occasions when a sample of animal material has to be collected.

The collecting may be required for example by particular events in the life of an animal such as its birth, the threat of an illness, its transportation or slaughter or again it may be collecting done periodically or else again collecting done after the animal's death, for example when material is collected from a portion of an animal intended for consumption or from leather.

These collecting operations can be done by the breeders themselves or by specific practitioners such as veterinary service staff, personnel in a slaughter-house or persons performing quality controls for an agri-food distribution undertaking.

To prevent risks of infection of the animal sampled or deterioration of the material collected, it is necessary to have aseptized equipment during these collecting operations. This includes not only the means for perforating the animal material that are in direct contact with the animal and/or the animal material, but also all the tools or means used during the collection, for example pliers or syringes, which need to be regularly disinfected.

This constraint is especially important for people working occasionally on several groups of livestock. It is indeed vitally important that they should not, through poor disinfection of a tool, introduce an illness from a previously visited herd into another herd.

It is also necessary to take care not to contaminate the collecting tools and, in particular, the components used to perforate the animal material when handling these components.

This is especially true when the person collecting the sample has not had lengthy practice in collecting operations and/or when the collecting is done on a living animal which is therefore likely to move during the collecting operation.

It is also important to limit the risks of injury to the practitioner during the collecting operation.

There is therefore a need for collecting equipment that is simple to use, offering guarantees of health and safety and especially of the safety of the practitioners.

### 3. SUMMARY OF THE INVENTION

The invention proposes a novel solution that does not have all these drawbacks of the prior art, in the form of a unit for collecting at least one sample of animal material, formed by a package comprising at least one collecting set.

According to the invention, such a collecting set comprises:

a collecting tool;

a component for collecting said sample;

a component for storing said sample;
at least one of said collecting or storing components being pre-mounted on at least one support provided for this purpose on said collecting tool.

The approach of the invention thus provides a breeder or any other user with the advantage of a “ready-to-use” device that is simple to use.

Furthermore, the solution enables a one-time use of the collecting tool, ensuring good hygiene for the collecting tool.

Said collecting set is intended for collecting any unspecified animal material, whether living or inert, for example animal tissue such as a piece of ear, skin, viscera or an animal fluid such as bone marrow or blood.

The collecting set of the invention can especially offer the possibility of providing several positioning supports. A support can for example be intended for the collecting component or another support can be intended for the storage component.

According to one particular characteristic of the invention, said at least one support defines at least one position for said collecting component and/or said storage component, belonging to the group comprising:

an initial position of transport;

a collecting position;

a final position of transport.

Thus, the solution of the invention, in providing for an initial position of transport of at least one of said components, makes it easier to transport the collecting unit, especially by reducing the space needed to stow it or by protecting certain fragile components.

The solution of the invention also offers the advantage, through a pre-mounted position of said collecting component in its collecting position, of limiting the handling errors during the preparation of the collecting, which could cause injury to the user or impair the quality of the collecting, owing to a poor installation of a component.

Finally, the presence of a transport support after the collecting has been done enables the person in charge of the collecting to have a practical means of stowing the collected sample and of thus limiting the risks of loss or deterioration of the sample.

According to one particular characteristic of the invention, at least one of said supports comprises a projecting feature enabling said animal material to be pushed into said storage and/or collection component after collection of said animal material.

According to one particular characteristic of the invention, said collecting tool is formed as one piece.

The solution of the invention thus makes it possible to propose a tool that is simple to make and costs little to manufacture.

The collecting tool can especially be made plastically, by injection-molding for example.

According to one particular characteristic of the invention, said collecting component is pre-mounted on said support in a collecting position, irreversibly.

Thus, the solution offers the advantage of permitting only a single use of the collecting tool. Thus, when the collection is done by a third party, for example a veterinary practitioner, the solution provides the breeder with a guarantee of the good hygiene of the collecting tool.

In addition, the solution of the invention also offers the advantage of reducing the number of operations needed for making a collecting operation in certain embodiments. It thus enables a user to be provided with a simpler operation of said tool. It also reduces the complexity of the collecting tool.

According to one particular characteristic of the invention, said collecting component is housed detachably in said storage component so as to protect said collecting component.

Thus, the solution of the invention prevents the collecting component from being capable of being damaged, for example being twisted, during the transporting of the collecting unit or from being capable of injuring the person who is transporting it or installing it, for example a breeder who might be transporting such a collecting unit in the pocket of a garment.

It also enables the collecting component to be preserved in a clean environment.

According to one particular characteristic of the invention, said collecting component comprises a cutting component designed to cut out said animal material and a pusher component enabling said animal material to be pushed into said storage component after said animal material has been cut out by said cutting component.

Thus, it is possible to preserve only the storage component once the collecting has been done.

This embodiment additionally offers the advantage of achieving a plugging of the storage component without any action being required by the user.

According to one particular characteristic of the invention, at least one of said collecting and/or storage components contains at least one specific agent belonging to the group comprising:

a disinfectant;

a cicatrizing agent;

a preserving agent;

a desiccant;

a medicine;

a vaccine;

a combination of at least two of the above agents.

Thus, the collecting or storage component may if necessary comprise means for processing the sample enabling its preparation, its preservation or again its analysis.

If the agent is a disinfectant or a cicatrizing agent, it enables the destruction of at least a portion of the microorganisms (bacteria, viruses, etc) that could contaminate the collected sample.

If the agent is a preserving agent or a desiccant, it improves the preservation of a sample of tissue to be collected and if necessary enables its preparation for subsequent treatment such as a DNA analysis.

If the agent is a medicine or a vaccine in contact with the collecting component, it enables the animal to be cured or treated if need be, this agent being capable of passing into the blood at the perforated tissues or being absorbed by the animal's skin.

According to one particular characteristic of the invention, said specific agent takes one of the forms belonging to the group comprising:

a gel;

a cream;

a grease;

a liquid;

a powder;

a gas;

an impregnated foam.

According to one particular characteristic of the invention, at least one collecting component consists of a biopsy needle, a perforating tool or a punch.

According to one particular characteristic of the invention, said collecting tool comprises means for closing said storage component.

The solution of the invention thus proposes an improved closing of the storage means: indeed, since the closing is done automatically, there can be no risk of negligence. In addition, since the closing means are adapted to the storage means, the result is generally more satisfactory than in the case of a manual operation where the operator will for example not venture to press too heavily on a plug, because of the risk of breaking a glass tube.

In certain embodiments where the closing means can prevent access to said storage component and/or collected animal material, without visible deterioration of said tool, the solution ensures that the animal material collected by the tool will really be the animal material that reaches the laboratory, should it be handled by third parties.

It also provides additional protection to the sample during transport, especially in the event of a suspected risk of an epidemic.

According to one particular characteristic of the invention, said collecting tool is a set of pliers or a syringe.

According to one particular characteristic of the invention, said collecting tool has a return means enabling the collecting tool to be brought back into an initial position once the collecting is done.

The term “initial position” is understood to mean the position of said tool prior the collecting operation.

Thus, the solution of the invention offers the advantage of more speedily releasing the animal material from which the collecting is done, this feature being especially useful when it is a living animal, for which the collecting operation is an act of aggression.

The return mans can be constituted especially by at least one leaf spring.

According to one particular embodiment of the invention, said collecting tool comprises at least two supports and at least one of said supports is intended for carrying at least one additional collecting and/or storage component.

Thus the solution for example offers a practitioner the advantage of being able to use the second collecting and/or storage component if the first collecting and/or storage component is soiled or deteriorated before the collection is made, for example because of a fall.

It also enable the performance of several distinct collecting operations with the aim of carrying out distinct tests or because a test requires several collecting operations, especially because it uses different specific agents.

For example, the solution enables a dual collecting operation comprising a first collection using a cicatrizing agent and then a second collection using another agent.

According to one particular characteristic of the invention, said collecting tool is at least partially recyclable.

Thus, the solution of the invention offers advantages in terms of sustainable development.

According to one particular characteristic of the invention, said collecting tool is at least partially designed in a material enabling a one-time use of said tool.

The term “one-time use” is understood to mean that the mode of manufacture and/or delivery of the collecting tool is adapted so that the collecting tool can be used only once. However, certain embodiments of the clamp enable the tool to be used a limited number of times (for example about 25 times).

According to one particular characteristic of c invention, said storage component contains a non-return means enabling said animal material to be held in said storage component when it is opened.

According to one particular embodiment of the invention, said collecting tool, said storage component, said collecting component and/or said package comprise means of identification enabling said collecting set and/or said collecting unit to be identified.

Said collecting unit, in certain embodiments where it contains only one collecting set, can have the same identification as the collecting set itself in other embodiments, for example embodiments in which the collecting unit comprises several collecting sets, the collecting unit and at least one collecting set that it contains can if necessary have distinct identifications. In this case, the packing of the collecting unit can for example comprise the identification of the collecting unit as well as those of the collecting sets that it contains.

The identification means may be visual means in the form of a sequence of numbers or a barcode, possibly a 2D code for example, or electronic means in the form of on RFID chip in particular.

Thus, for at least one of the components of a collecting unit, the solution makes it possible to identify the collecting unit and/or the collecting set of which it is a part and therefore to maintain traceability between certain components or even all the components of a same collecting unit.

In certain embodiments, the solution of the invention makes it possible for a user, at the time of the collecting, to associate the identifying of the collecting unit and/or the collecting set with the identification of the animal material collected, for example by means of a recording in a centralized database.

According to one particular characteristic of the invention, said identification means may contain means for identifying said collected animal material.

Thus, the solution enables a collecting unit to be associated with at least one animal as soon as it is manufactured.

According to one particular characteristic of the invention, said package is sterile.

Such a package thus enables the protection of a collecting set according to the invention. In particular, it enables to be preserved in a sterile environment. Consequently, it makes it possible to keep a collecting set sterile from its manufacture up to its use even if it is handled without precaution. It can also provide the set with thermal insulation, especially if the collecting set comprises a collecting or storage component which has been pre-filled with a specific agent. Finally, such a package enables the collecting set to be preserved from impacts or contact with other materials.

According to one particular characteristic of the invention, said package furthermore comprises an additional collecting or storage component.

### 4. LIST OF FIGURES

Other characteristics and advantages of the invention shall appear more clearly from the following description of a particular embodiment, given by way of a simple, illustratory and non-exhaustive example and from the appended drawings, of which:

FIGS. 1a to 1d illustrate a collecting set according to a first embodiment of the invention;

FIGS. 2 to 7 illustrate the collecting sets according to other embodiments of the invention.

### 5. DESCRIPTION OF ONE EMBODIMENT OF THE INVENTION

5.1 General Principle

The general principle of the invention consists in proposing a collecting unit comprising at least one all-in-one sterile collecting set, in a package, to a person in charge of carrying out collecting operations, for example a breeder, a veterinary practitioner, an employee of a slaughterhouse or an agri-food processing factory, etc., so that he has all the means needed to carry out at least one operation of collecting animal material and to carry out this operation of collecting in a very simple manner. A collecting set of this kind comprises especially a collecting tool (such as pliers or a syringe), a collecting component (such as a biopsy needle or a perforating tool), a storage component (such as a sampling tube) for storing the material collected. According to the invention, at least one of these collecting or storage components is pre-mounted on the collecting tool during the manufacture of said set.

According to one particular embodiment of the invention, the collecting set is planned for carrying out only one collecting operation. This embodiment makes it possible to prevent the contamination of a collecting tool during a previous collection. Once used, the collecting tool can be immediately discarded by the user or kept as a means for transporting the sample and then discarded by a third party. The collecting tool can especially be designed so as to be recyclable.

Certain embodiments of the collecting set enable a re-utilization of the tool. However, since the tool is not designed to withstand wear and tear, this re-utilization will be possible only in a limited way, for example in the range of 25 uses.

Since such a set is intended for a limited number of collecting operations, it offers the advantage of not requiring maintenance.

This leads to simplification from the user's viewpoint but also simplification of the mode of manufacture since, for example, the parts no longer need to be capable of being dismantled.

Yet another advantage of the invention, resulting from the absence of any constraint of resistance to wear and tear, is that use of a material that is lighter and/or equipment that is smaller sized equipment than in the prior-art solutions makes it possible to propose a lighter and, hence, easier-to-use tool. For example, the collecting tool can be made out of plastic.

Yet another advantage of the invention, owing to the equipment used and the simplicity induced in the making of the collecting tool is the low cost of manufacture of the collecting unit. This low cost enables a user to frequently change his collecting tool because the previous one is no longer usable or because he wishes to have a more recent model.

On the contrary, the prior-art collecting tools are often costly because of their complexity and because of the materials implemented, leading a breeder to have a single set of pliers which can be shared by several users and thus increasing hygiene-related problems.

The invention, on the contrary, gives the breeder the possibility of acquiring several sets of pliers, each set of pliers being allocated for example to one particular user.

The invention also enables thereby a major gain in time, especially when a collecting operation needs to be performed on each head of a large herd of livestock since several collecting operations can be done at the same time. This speed of collecting can furthermore give rise to speed of diagnosis in the event of an epidemic.

One of the advantages of the solution of the invention also flows, in certain embodiments, from the absence of means for simultaneous tagging (enabling the placing of an identification tag).

Indeed, the collecting set in at least one embodiment of the invention offers the advantage wherein it can be used on an animal already having an identification tag. Indeed, if the animal is already tagged, the collecting of a sample of animal material using a device that simultaneously performs identification and collecting would result in the assigning of a new identifier to a same animal. The solution of the invention on the contrary offers the breeder the advantage of having a solution that enables repeated tests on a same animal.

Another advantage of the device of the invention is that it can be adapted to DNA sampling. Indeed, DNA tests are costly. A breeder therefore has interest in carrying out this test not at the time of the birth of the animal (where it has to be identified) but when the animal proves to be viable and when the breeder knows the use to which he is going to put it.

5.2 Examples of Collecting Units According to the Invention

A collecting unit according to the invention is formed by a package comprising at least one collecting set.

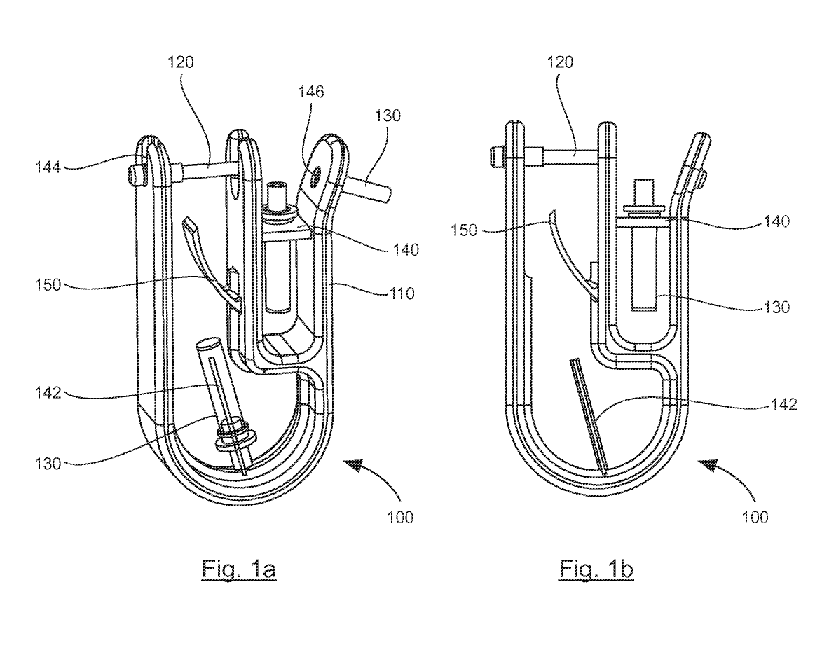

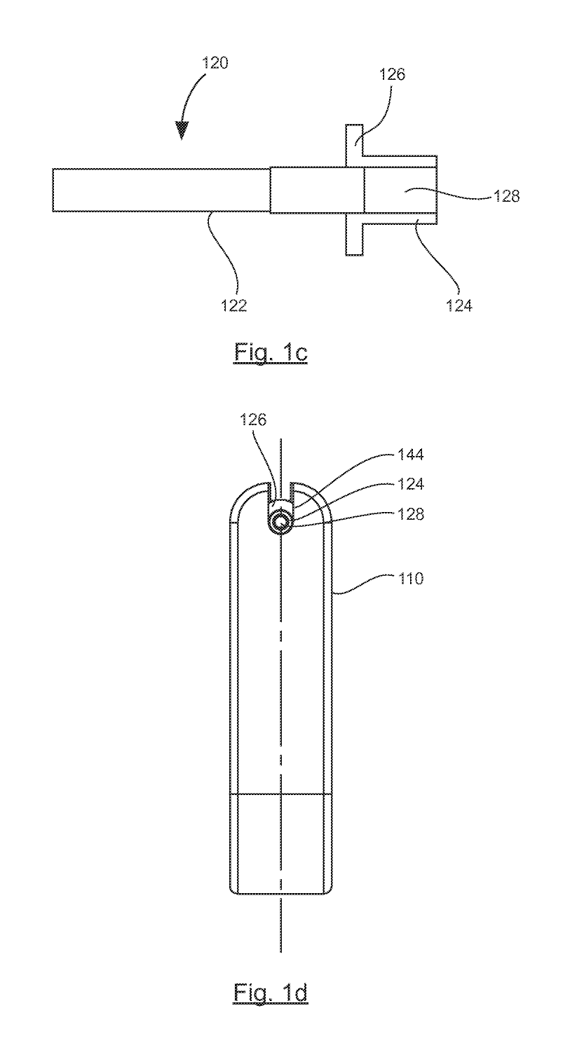

Here below, referring to FIGS. 1a to 1d, we describe a first embodiment of a collecting set according to the invention used for example to collect a sample of an animal's ear,

Thus, as illustrated in FIG. 1a, a collecting set 100 of this kind comprises a collecting tool which may especially take the form of a set of pliers 110.

It also comprises a collecting component 120 and a storage component 130 as well as supports 140, 142 and 144 situated on the collecting tool and intended for carrying the collecting component 120 and/or the storage component 130.

5.2.1 Collecting Component

Referring to FIG. 1c, a view in section is presented of the collecting component 120.

This collecting component has a cutting component with a cutting edge.

In the embodiment shown, the cutting component has a perforating tool 122 mounted on a rigid and hollow support 124 which can be made out of plastic, comprising a peripheral flange 126.

The support 124 has a cavity 128 designed to cooperate with a pin of the collecting tool 120.

If the cutting component is inserted into the storage component 130 (before or after the collecting operation), the flange 126 of the cutting component 122 can serve as a stop to limit the covering of the cutting component 122 by the storage component 130 and thus create a space between the end of the storage component 130 and the cutting component 122.

This space can especially enable the collected sample to be housed in the storage component 130.

According to one variant, the cutting component 122 and the support 124 are formed as a single piece, for example made of plastic or metal.

According to another variant, a collecting component can also comprise a pusher component which is mobile relatively to the cutting component as described in the French patent application filed under number FR-08 58453, not published, enabling the animal material to be pushed into the storage component after being cut out.

5.2.2 Storage Component

As illustrated in FIG. 1a, the storage component 130 consists for example of a tube capable of at least partially containing the collecting component, so as to cover the cutting end of the perforating tool and isolate the collected animal material from the external environment.

The collecting component 20 then closes the storage tube 130 in a tightly sealed or almost tightly sealed way.

According to one variant, the animal material is pushed into the storage component after collection. The storage component can then be closed by a plug or by a component of the collecting tool or by the pusher component described here above, etc.

In particular, the storage component may comprise a tube top piece comprising flange taking support on the edge of the storage tube. It can also contain retaining means enabling the collected animal material to be preserved in the storage component when it is opened.

5.2.3 Collecting Tool

In certain embodiments of the invention, the collecting tool 110 is least partially designed out of a material enabling a one-time use of the tool 110. In particular, the embodiment illustrated in figure la provides for the cutting tool 110 to consist of one piece obtained for example by injection molding of a plastic material.

The invention also provides for the collecting tool 110 to be at least partially recyclable.

In the embodiment described in FIG. 1a, the collecting tool 110 is formed as one piece and takes the form of a set of pliers comprising two arms or grips of any kind whatsoever.

A pressure, for example a hand pressure, on the arms, guides the collecting component so that it penetrates the animal's flesh and forces the collected animal material to enter the storage component.

The pliers 110 can be actuated also electrical, pneumatic or other energy.

According to the invention, the collecting tool 110 comprises at least one support for positioning the collecting component 120 and/or the storage component 130. These supports can especially enable the collecting component 100 of the storage component 130 to be placed in various positions:

an initial transport position from the time of manufacture of the collecting set to the performance of the collecting operation,

a positioning for collecting, or again

a final position of transport after the collecting operation until it is conveyed into a laboratory for example.

For example, FIG. 1a illustrates the different positions of the collection or storage components implemented, successively:

before the collecting set is packaged to form the collecting unit, the collecting component 120 can be inserted into the storage component 130, held in a first support 140 of the collecting tool 110, taking for example the form of an aperture into Which the storage component 130 is inserted. This first support 140 defines an initial position of transport.

during the collecting operation, the collecting component 120 is positioned on a first jaw of the tool, in a second support 144 and the storage component 130 is positioned in a second jaw of the tool in a third support 146. These two supports 144 and 146 define a collecting position;

after collection, the collecting component 120 can be inserted into the storage component 140 and held on a fourth support 142 taking for example the form of a rod. A rod of this kind can especially get inserted into the collecting component by its end opposite the cutting edge at the cavity 128 and push the collected material into the storage component. The cavity 128 can for example be plugged by means of a foam-type permeable material or perforated with a narrow aperture. This fourth support 142 defines a final transport position.

These positions can be distinct, as illustrated by the supports 140, 142, 144 and 146 of FIG. 1a, or they can coincide.

In particular, the initial and final positions of transport can coincide.

According to one variant, the collecting and storage components are directly pre-mounted in a collecting position before the collecting unit is packaged to form the collecting unit.

The invention provides for different modes of fixedly joining the collecting components or storage components to the support.

For example, in the embodiment illustrated in FIG. 1d, the collecting component 120 is fixedly joined to the second support 144 in the collecting position by means of its flange 126.

According to at least one embodiment, the invention provides that at least one of the supports will comprise a projecting feature enabling the animal material to be pushed into the storage component and/or into the collecting component after the animal material has been collected when the storage component and/or the collecting component is positioned on the support. This is for example the rod of the fourth support 142 illustrated in FIGS. 1a and 1b which makes it possible especially to push the collected sample to the bottom of the storage tube.

The presence of a projecting feature of this kind on a support can also make it possible to close a storage component positioned on this support.

In one variant of the embodiment presented by FIGS. 1a to 1d, the collecting tool comprises several supports to carry at least one additional collecting or storage component, for example so as to enable a replacement of the collecting or storage component in the event of a defect or soiling. Thus, several collecting and/or storage components can be present simultaneously on the collecting tool.

These multiple collecting and/or storage components can also make it possible to carry out several collecting operations with a same collecting tool for example for a test requiring several collecting operations or for distinct tests.

In certain embodiments of the invention, the collecting tool 110 also has a return means by which the collecting tool can be brought back into its initial position once the collecting is done. These return means can, for example, consist of a spring or, as shown in FIG. 1a, a flexible tab 150.

In one particular embodiment compatible with the embodiment presented in FIG. 1a, the collecting component and/or the storage component can contain at least one specific agent aimed at the preparation the preservation or again the processing of the sample.

It may for example be a disinfectant, a cicatrizing agent or a desiccant such as a silicagel (registered mark) or more generally a molecular sieve based on silica, clay or the like, a preserving agent, a medicine or a vaccine or again a combination of at least two of the foregoing agents.

Various types of product taking the form of balls, powder, gel, cream, grease, liquid, cream, gas, etc, possibly impregnated in a foam, can be used.

In other words, the specific agent according to this embodiment of the invention can take the form of any product capable of acting on animal material that is collected, or of acting on the collected sample.

5.2.4 Collecting Unit

According to the invention, the collecting set 100 is conditioned in a package thus forming a collecting unit.

According to the invention, the packaging of the collecting unit and/or the collecting set 100 itself may comprise identifying means. These may be visual identifying means taking the form of a sequence of digits or a barcode, possibly a 2D code, or an electronic code in the form of an RFID chip especially.

For example, a same identifying number is allotted to the collecting tool, the collecting component, the storage component and/or the packaging. These identifying means thus enable the identification of both the collecting unit, the collecting tool, the collecting component and the storage component. They can also make it possible to characterize the collecting done, in particular through the timestamp and place of the collecting operation, and the type of animal material collected.

In one particular embodiment, these identifying means may be combined with means for identifying animal material, for example to enable a traceability of the collecting done, or of at least one part of the collecting set used with the animal material on which the collecting is done.

The collecting set 100 can be conditioned in a sterile environment, for example a sterile package or liquid, to form a collecting unit so as to be easily transportable. Depending on the embodiments, the packaging can be hermetically sealed, for example in the form of a bag, or may take the form of a sheet, especially a sheet of a specific paper. In particular, it may be a package permeable to certain gases filled with a sterilizing gas after the collecting set has been enveloped.

In one particular embodiment of the collecting unit, the collecting set is thus enveloped in a package of this kind and then placed in a climatic chamber which is then filled with ethylene oxide in order to obtain a sterile space around the collecting set.

In other embodiments of the collecting unit, possibly complementary embodiments, the collecting set can be delivered in an isothermal or hermetically sealed package or a package for protecting it against impacts.

Such a packet may furthermore comprise other means necessary for collecting, especially an additional collecting set and/or at least one additional collecting or storage component, a container of disinfectant, means for processing the sample enabling its preparation, preservation and/or processing or a piece of equipment aimed at better hygiene for the user.

The approach of the invention therefore enables the user to have a ready-to-use collecting unit containing both the equipment needed for collecting as well as other supplementary means such as disinfectant or gloves or disposable caps.

In one alternative embodiment, the package may also include several collecting sets, some comprising for example collecting tools of different shapes (for example of different sizes).

Thus, the approach of the invention enables the user to have several tools at a time and to choose the one best suited, depending for example on the type of animal from which material is to be collected, the collecting to be done, the dangerousness of the animal, etc.

5.2.5 Method For Making a Collecting Unit

According to the invention. several embodiments of the collecting unit can be implemented.

Thus, in one particular embodiment, the method for making collecting unit comprises:

a step for manufacturing at least one collecting component and/or at least one storage component;

a step for making at least one collecting set, during which at least one of said collecting components and/or said storage components is pre-mounted on a collecting tool;

a step for constituting the collecting unit during which the pre-mounted collecting set is conditioned in a package.

In certain embodiments, the step for making the collecting set comprises firstly a step for manufacturing the collecting tool and secondly a step for mounting said collecting component and/or said storage component on said collecting tool.

In other modes of manufacture, which may be complementary modes, the step for mounting the collecting component and/or the storage component can be performed during the step for manufacturing the tool. This embodiment is also suited for example to a mode of manufacture of a one-time-use collecting tool where the collecting component and/or the storage component is fixed into the collecting tool for example so that it is impossible to remove the component without damaging the tool.

According to the invention, the collecting component and/or the storage component can be positioned on the tool during the step for making the collecting set independently of one another, or associated with one another.

Thus, in the particular embodiment illustrated in FIG. 1a, the collecting component 120 is inserted into the storage component 130 during the step for making the collecting set 100. The collecting set is then packaged to form the collecting unit during the step for constituting the collecting unit.

This embodiment prevents the user from getting injured during the handling of the collecting component and protects the edge of the cutting component of the collecting component from the external environment. It also protects a specific agent, which may be provided in the hollow part of the collecting component, from the surrounding air.

5.3 Presentation of a Second Embodiment

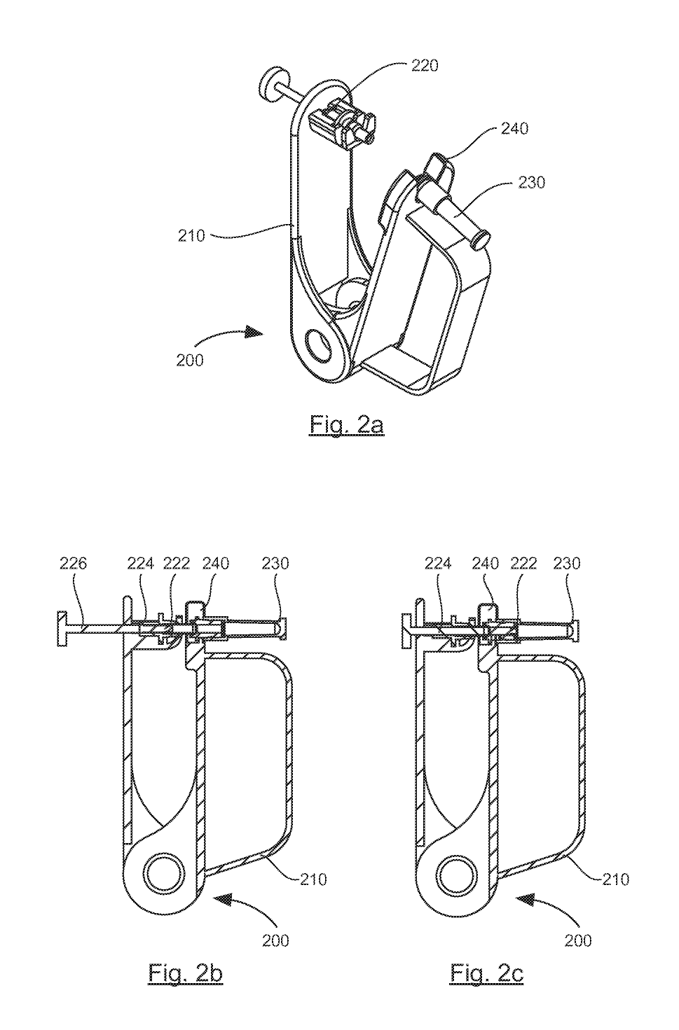

Referring to FIGS. 2a to 2c, we present a second embodiment of a collecting set 200 of a collecting unit according to the invention, in which there is again a collecting component 220, a storage component 230 and a collecting tool 210 comprising at least one support 240 of the collecting component 230.

According to this embodiment, the storage component 230 is pre-mounted on the support 240 before the collecting set is packaged to form the collecting unit. The collecting component 220 can also be pre-mounted on a second support or inserted into the storage component 230 for transport to the user.

In this embodiment, as illustrated in FIGS. 2b and 2c, the collecting component 220 may, in addition to the cutting component 222, which plays a role similar to the cutting component 122 shown in FIG. 2c, include a pusher component 224 actuated by a pin 226.

As presented in FIGS. 2b and 2c, the cutting component 222 is open at both ends in order to let through the pusher 224 so that this pusher can push the sample of animal material out of the cutting component 222 and accompany it into the storage component 230 and plug the storage component 230.

Once the sample has been pushed into the storage component, the collecting component 220 used can, depending on the embodiments, be preserved with the storage component or discarded.

In one variant, the pin 226 can furthermore include return means, for example a spring, in order to enable a speedier and automatic disengagement of the tool from the perforated zone and therefore, in a collecting operation performed on a live animal, a faster releasing of the animal.

As can be seen in FIG. 2a, the closing of the tool is done by rotation of at least one of the arms of the tool and therefore gives a curved motion to the path of the collecting component. This first rotation motion enables the cutting component 222 to cut the animal matter. In a second stage, the pin 226 is actuated in a translational motion, enabling the pusher component 224 to push through the cutting component 226 as described in the above-mentioned patent application FR-OS 58453.

5.4 Presentation of a Third Embodiment

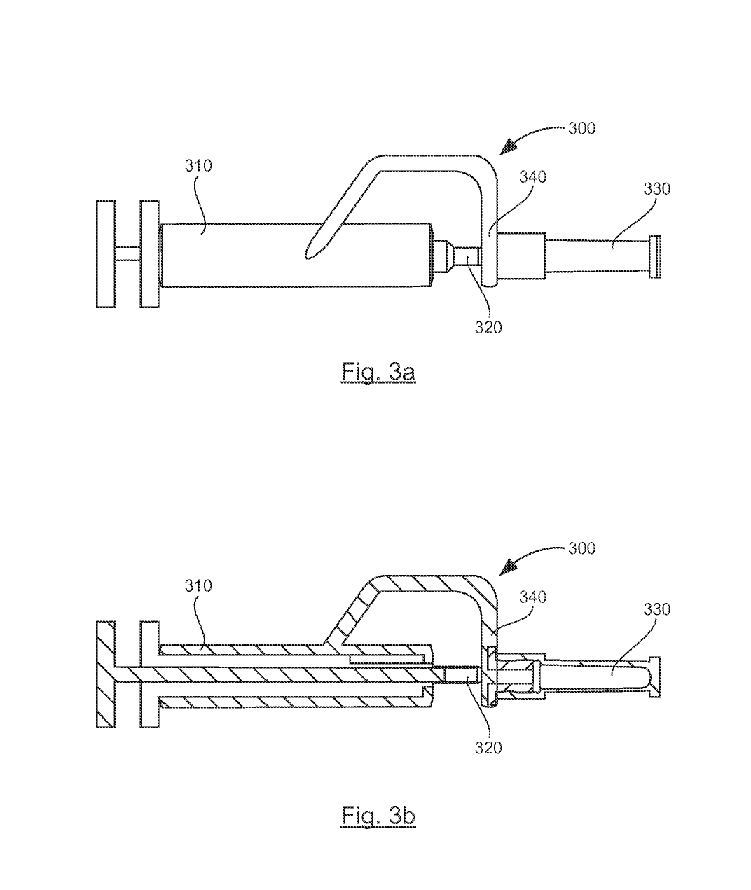

Referring now to FIGS. 3a and 3b, a third embodiment of a collecting set 300 of a collecting unit according to the invention is presented, wherein there is again a collecting component 320, for example a biopsy needle, a storage component 330 and a collecting tool, for example a syringe 310 comprising at least one support 340.

FIG. 3a presents a front view of a collecting set 300 and FIG. 3b shows the cross-section.

The storage component can be mounted non-detachably, at the very time of manufacture of the collecting set 300, in a collecting position by means of the support 340.

This embodiment makes it possible to obtain a one-time use of the collecting tool 310.

In this embodiment, the tool 310 imparts a motion of translation to the collecting component 320, enabling higher precision of collection. Once the collecting is done, the storage component can be closed by a plug or by the collecting component (after the releasing of the animal's ear for example) and the syringe can be forwarded directly to a laboratory for tests.

In one variant of this embodiment, where the collecting component can be withdrawn from the support 340, the end not intended for collecting can for example be ended by a plug so as to close the storage component.

5.5 Presentation of a Fourth Embodiment

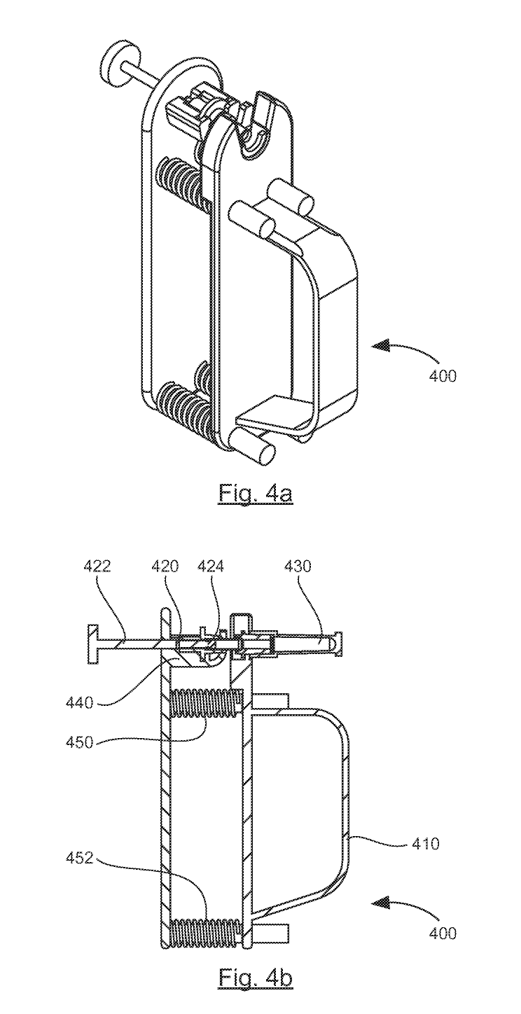

Referring to FIGS. 4a and 4b, we present a fourth embodiment of a collecting set 400 of a collecting unit according to the invention, wherein there is again a collecting component 420 which may include a pusher component 424 in addition to the cutting component 422, which plays a role similar to the cutting component 122 presented in FIG. 2c.

There is also a storage component 430, and a collecting tool, for example a plier 410 comprising at least one support 440.

According to this embodiment, the collecting component 420 is pre-mounted on the support 440 before the collecting set is packaged to form the collecting unit. This storage component 430 can also be pre-mounted on a second support or inserted on the collecting component up to the use.

For example, the storage component consists of a storage tube as described in the above-mentioned patent application FR-08 58453.

FIG. 4a shows a view in perspective of the collecting set 400 and FIG. 4b shows a view in section.

In the example illustrated, the closing mechanism of the pliers 410 and especially the springs 450 and 452 impart a translational motion to the collecting component. In this way, the path of the cutting component 422 and of the pusher element 424 through the animal material to be perforated is rectilinear, enabling higher precision of the collecting operation.

This first translational motion enables the cutting component to cut into the animal material. In a second stage, a pin 426 is actuated in a second translational motion, enabling the pusher 424 to be pushed through the cutter 422.

FIGS. 4a and 4b provide an exemplary embodiment implementing four springs. Other recall means or a different number of springs (two springs for example) can also be envisaged.

5.6 Presentation of a Fifth Embodiment

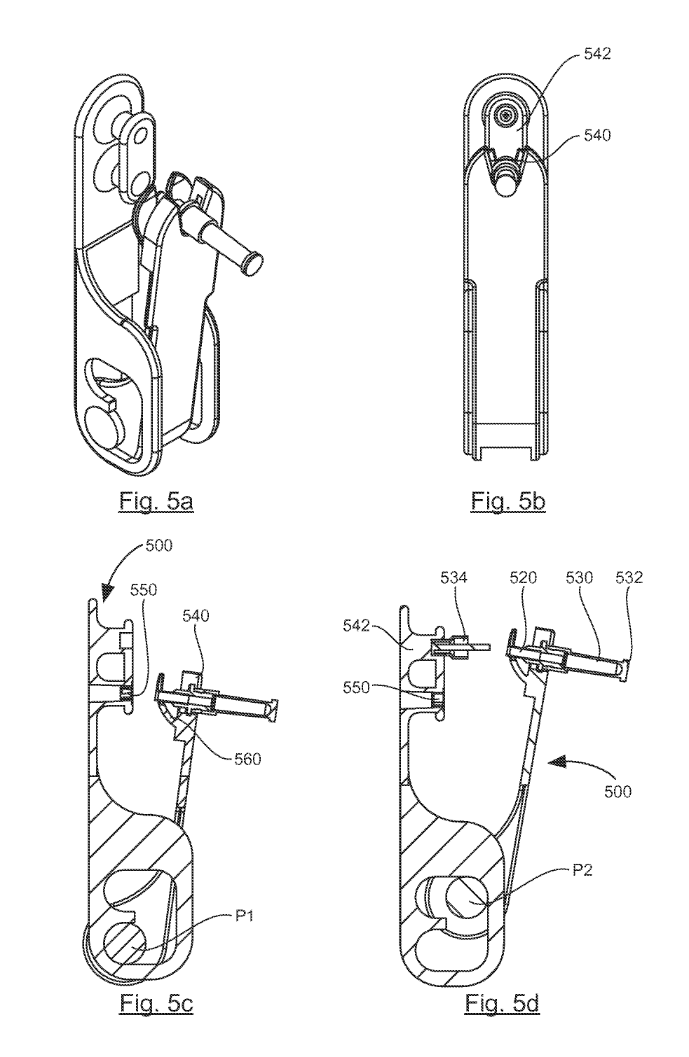

Referring to FIGS. 5a to 5d, we present a fifth embodiment of a collecting set 500 of a collecting unit according to the invention wherein there is again a collecting component 520, a storage component 530 formed by two parts, for example a storage tube 532 and a plug 534, and a collecting tool 510.

The collecting tool 510 comprises a first support 540 designed to receive the collecting component associated with the storage component.

According to this example, the collecting component 520 is a through component and is inserted at least partially in the storage component 530.

FIG. 5a shows a front view of the collecting set 500, FIG. 5b shows a top view and FIGS. 5c and 5d show cross-sections at different instants in the collecting operation.

As illustrated by these figures, the collecting tool comprises a recess 550 on which there rests the cutting component of the collecting component so that it can more easily perforate the animal matter during the collecting operation.

The collecting tool 510 also has a plug 534 on a support 542. Such a plug comprises a rod used to push the animal material collected through the collecting component up to the storage component 530.

The collecting tool is provided with a rotation spindle with two positions P1 and P2 which are used to modify the position of the collecting component during the closing of the clamp. Thus, a first position P1 of the spindle places the collecting component 520 and the recess 550 face-to-face to carry out the collecting operation. A second position P2 of the spindle places the collecting component 520 and the plug 534 face-to-face so as to push the sample of material into the storage tube 532 and close the end of the collecting component 520.

A leaf spring illustrated by the flexible tab 560, can also be provided.

This leaf spring also makes it possible to push back the perforated animal material, for example an animal's ear, and move the collecting component away from the hole made so that the collecting component no longer “clings” to the perforated animal material, especially an ear, and to facilitate the return of the collecting tool into the initial position once the collection has been made.

5.7 Presentation of a Sixth Embodiment

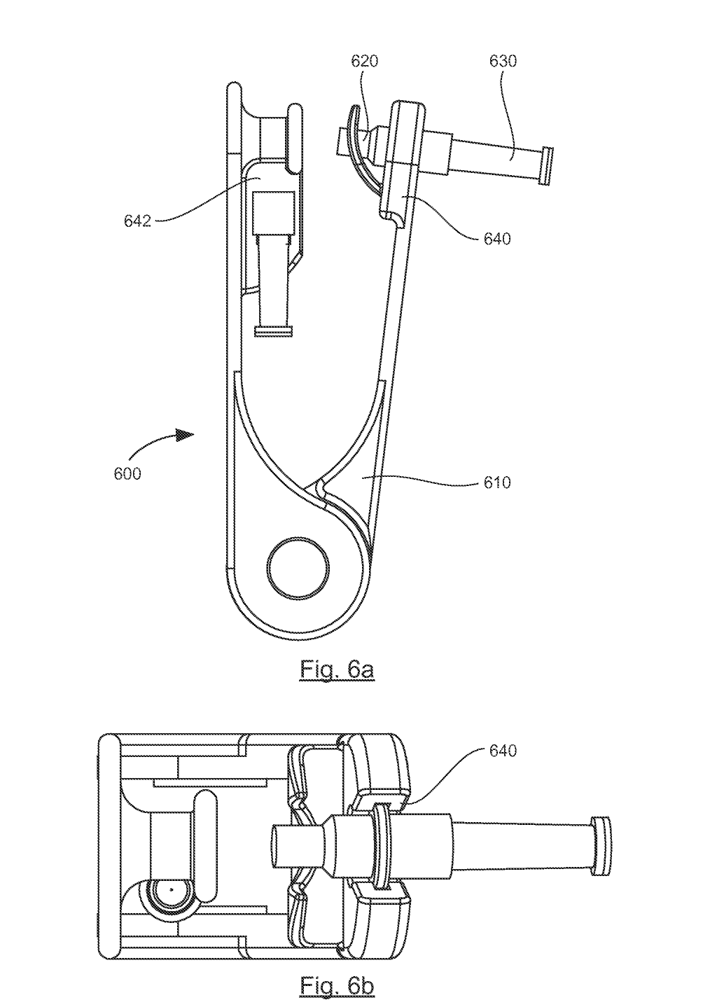

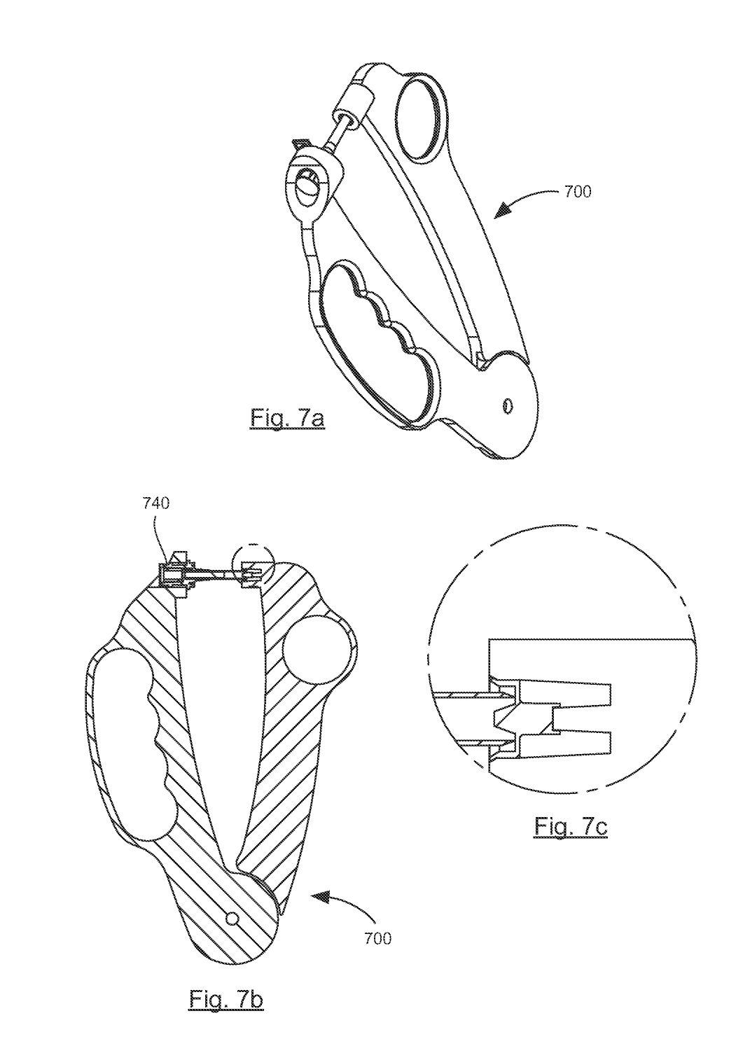

A sixth embodiment is presented of a collecting tool 600 of a collecting unit according to the invention with reference to FIGS. 6a and 6b which respectively present a view in profile and a top view thereof.

In this collecting set 600, there is again a collecting component 620, a storage component 630 and a collecting tool 610 comprising a first support 640 to position the collecting component, for example associated with the storage component, in a collecting position.

According to this example, the collecting component 620 is a through component and is at least partially inserted into the storage component 630.

The collecting tool comprises for example two arms, a first arm on Which the storage component and the collecting component are pre-mounted and a second arm comprising a recess on which there rests the cutting component of the collecting component so as to perforate the animal material more easily during the collecting operation.

The collecting tool 610 also comprises a second support 642. This second support can define an initial or final position of transport for the collecting component and/or the storage component.

According to one variant, this second support enables a second back-up storage component to be positioned.

A leaf spring can also be provided, it makes it possible especially to push back the perforated animal material, for example an animal's ear, and to move the collecting component from the hole made, so that the collecting component does not “cling” to the perforated animal material, especially an ear, and to facilitate the return of the collecting tool to its initial position once the collection is done.

5.8 Presentation of a Seventh Embodiment

A seventh embodiment is presented of a collecting set 700 of a collecting unit according to the invention, with reference to FIGS. 7a to 7c which present a view in perspective, a cross-section view and the open end of the collecting component 720.

In this collecting set 700, there is again a collecting component 720 and a collecting tool 710 comprising a support 740 to position the collecting component 720 in a collecting position. The support 740 is provided on a first jaw of the tool. A projecting feature provided on a second jaw of the tool pushes the animal material back into the collecting component.

Thus, a partial closing of the collecting tool enables the collecting to be done.

A complete closing of the tool (after the withdrawal from the animal's ear for example) enables the animal material to be pushed into the collecting component.

The collecting component can then be dismantled from the tool and inserted into a storage component that is not shown.

In one variant, the tool is transmitted to the laboratory after complete closure. In this case, the collecting component 720 can also enable the storage of the collection made. The complete closing of the tool therefore enables the collecting component to be isolated once the collecting has been done.

In one variant of this embodiment, the collecting component 720, which also fulfils the role of a storage component of the collection made, is fixedly joined irreversibly to the collection tool 710 by means of the support 740.

5.9 Other Embodiments

The embodiments of the invention are not limited to the embodiments presented herein. Other embodiments, intuitively known to those skilled in the art from the reading of the description of the invention, are possible. For example, embodiments presented, or options pertaining to these embodiments, such as the presence of several supports or return means can especially be combined with one another.

In another variant of the invention, the collecting tool comprises means for closing the storage component. These for example may be a simple plug as illustrated in FIGS. 5a to 5d, or means preventing access to the storage component such as for example means enabling it to be sealed or enabling the tool itself to be sealed if the component is carried on a support of the tool in a final position of transport.

In this way, once the collecting operation is performed, it is possible to directly send the collecting tool to a laboratory in order to remove it and carry out tests on the animal material. This operation is possible because of the one-time use for which the collecting set of a collecting unit according to the invention is intended.

According to another embodiment, the tool of the collecting set presented could be coupled with another device or could possess additional means, enabling especially the consecutive or simultaneous positioning of an animal identification tag. In particular, this identification tag could carry an identification related to an identification means of the collecting set and/or the collecting unit.

Furthermore, one variant of the invention provides that the collecting tool can be installed on another device, for example a tagging device, through which it is actuated.

In all the embodiments presented, the collecting set is intended for one-time use and could therefore be discarded and, in particular, it could be at least partially recycled after a one-time use.

Indeed, the tool is not necessarily made out of aluminium or steel but can be made of plastic or another light material which does not have the same longevity as the collecting tools of the prior art.

In certain embodiments, the collecting tool is designed so as to be almost flat or of a small thickness in order to facilitate its transport or especially its dispatch by postal means before or after the collecting operation.

The low cost of making the collecting tool indeed makes it possible to propose a discardable tool.

In all the embodiments presented, the packaging of the collecting unit containing the collecting set is liable to be sterile and especially hermetically sealed or enabling protection against impact, for example as a padded bag suited to being sent by post. It may also be a sterile package in the form of at least one sheet enveloping the collecting set and filled with a sterilizing gas. The packaging itself can be recycled or designed to be re-utilized, especially for to transport the storage component once the collecting has been done.

1. A collection unit for collecting at least one sample of animal material, consisting of a package comprising:
at least one collection set;
wherein a collection set comprises:
a collecting tool;
a collecting component configured to collect a sample of animal material; and
a storing component configured to store said sample of animal material:
wherein at least one of said components for collecting or storing being pre-mounted on at least one support provided for this purpose on said collecting tool.

2. The collection unit according to claim 1, wherein the at least one support defines at least one position for at least one of said collecting component or said storing component, selected from the group consisting of an initial position of transport, a collecting position, and a final position of transport.

3. The collection unit according to claim 1, wherein at least one of said supports comprises a projecting feature enabling said animal material to be pushed into said storing or collecting component after collection of said animal material.

4. The collection unit according to claim 1, wherein said collecting component is pre-mounted on said support in a collecting position, irreversibly.

5. The collection unit according to claim 1, wherein said collecting component is housed detachably in said storing component so as to protect said collecting component.

6. The collection unit according to claim 1, wherein said collecting component comprises a cutting component designed to cut out said sample of animal material and a pusher component enabling said sample of animal material to be pushed into said storing component after said animal material has been cut out by said cutting component.

7. The collection unit according to claim 1, wherein at least one of said collecting or storing further comprises at least one agent selected from the group consisting of a disinfectant, a cicatrizing agent, a preserving agent, a desiccant, a medicine, and a vaccine.

8. The collection unit according to one of the claim 1, wherein said collecting tool comprises means for closing said storing component.

9. The collection unit according to claim 1, wherein said collecting tool comprises a return means enabling the collecting tool to be brought back into an initial position once the collecting is done.

10. The collection unit according to claim 1, wherein said collecting tool comprises at least two supports and in that at least one of said supports is intended for carrying at least one additional collecting and/or storing component.

11. The collection unit according to one of the claim 1, wherein said collecting tool is at least partially recyclable.

12. The collection unit according to claim 1, wherein said collecting tool is at least partially designed in a material enabling a one-time use of said tool.

13. The collection unit according to claim 1, wherein at least one of said collecting tool, said storing component, or said collecting component further comprises means of identification enabling said collection set to be identified.

14. The collection unit according to claim 1, wherein said package is sterile.

15. The collection unit according to claim 1, wherein said package furthermore comprises an additional collecting or storing component.
